(19) United States
(12) Patent Application Publication
(10) Pub. No.: US 2021/0401331 A1
(43) Pub. Date: Dec. 30, 2021

(54) Glucose Monitoring Smart Watch

(71) Applicant: Belinda Flores, Kissimmee, FL (US)

(72) Inventor: Belinda Flores, Kissimmee, FL (US)

(21) Appl. No.: 16/917,880

(22) Filed: Jun. 30, 2020

(51) Int. Cl. A61B 5/145; A61B 5/15; A61B 5/00

(52) U.S. Cl. CPC A61B 5/14532; A61B 5/150305; A61B 5/747; A61B 5/0004; A61B 5/0022; A61B 5/681

(57) ABSTRACT

The Diabetic Glucose Monitoring Smart Watch, in conjunction with the “Save a Life, Link a Buddy” smart phone application, is designed for any person with Type I or Type II Diabetes. The smart watch will monitor the blood glucose levels of the wearer through a continuous glucose monitor and will alert the user in real-time if glucose levels reach a danger zone designated by the user during set up. If the wearer is unresponsive to the alert for over 60 seconds, the linked application (“Save a Life, Link a Buddy) will then notify the “buddies” assigned by the user. This alert will appear on the “buddies” phone similar to an Amber-Alert that needs to be turned off manually.

### BACKGROUND OF THE INVENTION

### Field of the Invention: Medical

Glucose Monitoring Smart Watch—Was previously invented but was not accurate. After speaking with an endocrinologist, I modified the invention. I was able to use real life experience of what is needed because two of my children are Type I Diabetic.

### BRIEF DESCRIPTION OF DRAWINGS

FIG. 1:

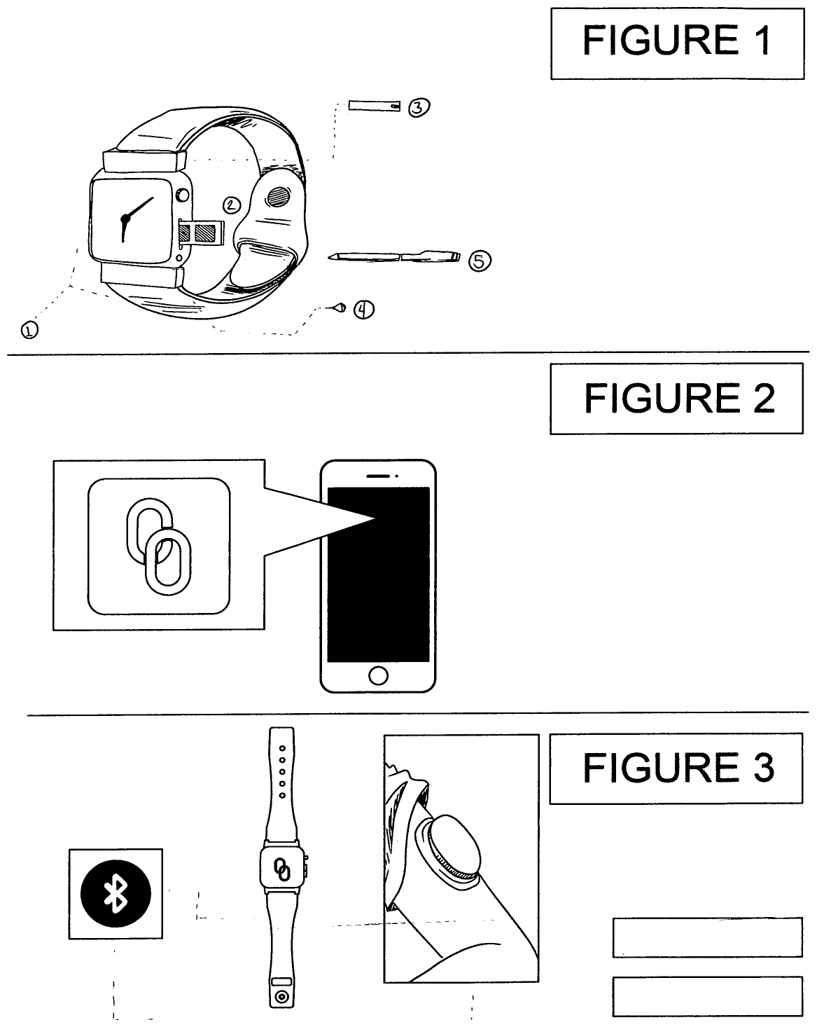

Feature 1—Is the Diabetic Glucose Monitoring Smart Watch. This is a standard smart watch with a glucose meter functionality. Users will be able to test their glucose levels using the watch to display their glucose readings in real time throughout the day.

Feature 2—Is the compartment in the base of the watch that will allow for a strip to be inserted for accurate glucose readings.

Feature 3—Is the strip itself that will inserted in the he storage compartment is located between the wristband and base of the watch.

Feature 4—is the Lancet. This is located opposite of the strip compartment and will balance out the overall layout of the watch.

Feature 5—is the lancing device with interchangeable lancets that is stored and located on the base of the watch. Similar to a smart phone pen.

FIG. 2:

Phone Application—The Phone application will need to be installed and will connect with the watch via Bluetooth. The application will allow the user to customize their target range for better A1C results as well as analyze and monitor their overall health and fitness.

FIG. 3:

Sensor—The Sensor is an optional feature that will allow for better accuracy when displayed the glucose levels through the day. This will connect to the watch as well as the phone application via Bluetooth.

### BRIEF SUMMARY OF THE INVENTION

### Glucose Monitoring Smart Watch Benefits

Controlled Blood Glucose levels with a monitoring system
Target ranges for monitoring blood glucose levels
Alerts when glucose levels reach dangerous levels
Alerts when sensor needs to be replaced
Glucose readings displayed at all times
Linked to an individual(s) of choice
Activity monitoring system
Medical Team dispatch
GPS location device
A1C Estimator

### DETAILED DESCRIPTION OF INVENTION

### Detailed Description of Diabetic Glucose Monitoring Smart Watch

This product doubles as a smart watch and a continuous glucose monitor
The band of the smart watch will have storage for the glucose testing strips immediately above the face of the watch
The band of the smart watch will have storage for the lancet (which pricks the user to draw blood to use on the glucose testing strips) directly below the face of the watch
The right side of the face of the watch will have an opening for the user to insert the glucose testing strips, the watch will be able to read the glucose levels of the user's blood the same as a glucose monitoring meter. This technology will be built into the watch.
The watch will then monitor the user's glucose levels for approximately 12 hours through sensor technology and known algorithms that will monitor items such as fitness, sweat production, shaking, heart rate and continuously update the blood glucose levels of the users on the watch.
The user will also have the ability to link other apps to log carbohydrate intake such as MyFitnessPal.
The user also has the option to connect their Diabetic Glucose Monitoring Smart Watch to their continuous glucose monitor (ie. The Libre) through a bluetooth connection. This option will provide the most accurate, real-time readings.

The “Save a Life, Link a Buddy” app will need to be downloaded by both the watch user and their assigned buddies. The application will run in the background and will be the home application that stores the users blood glucose history. Within the application, the user can assign “buddies,” which will then link the user and their buddies by phone number to allow the application to send alerts to all linked “buddies” if glucose levels drop or heighten to dangerous levels. During set up, the user will be required to set up the following specifications:
Link the application to their smart watch
Allow the application permission to transmit data (blood glucose levels) from the smart watch, allow sharing of location and to allow push notifications
Allow the app constant access to their location
Set their designated levels of “dangerous” blood glucose levels (both too high and too low)
Insert their medical information (age, sex, weight, insurance and medication information)
Assign “buddies”—individuals who also have the application downloaded on their phone. These buddies will then receive alerts when the wearer of the watch has blood glucose levels that reach dangerous levels and has not responded to their own alert from the app for over 60 seconds

“Buddies” will be required to set up the following specifications:
Allow the applications to send push notifications to their smart phone
Accept “buddy” requests if they want to receive notifications from a user
   How the “Save a Life, Link a Buddy” works:

If the user's glucose levels reach their designated “danger zone” the user will be presented with pulse checks to ensure they are conscious. The pulse checks will be in the form of simple puzzles that will appear on the user's smart phone (via the app). If the user cannot solve the puzzles or does not respond to the alert within 60 seconds their “buddies” will be sent a notification to their phone along with the user's GPS location. The designated buddy will be presented the option to call paramedics immediately or attend to the user if they are within a certain distance (less than 5 miles). If their glucose levels reach danger zones/levels for over 5 minutes and no “buddies” note that they have responded in the app, paramedics will automatically be contacted with all of the user's medical information and their location.

### SPECIFICATION

The Diabetic Glucose Monitoring Smart Watch, in conjunction with the “Save a Life, Link a Buddy” smart phone application, is designed for any person with Type I or Type II Diabetes. The smart watch will monitor the blood glucose levels of the wearer and will alert the user in real-time if glucose levels reach a danger zone designated by the user during set up. If the wearer is unresponsive to the alert for over 60 seconds, the linked application (“Save a Life, Link a Buddy) will then notify the “buddies” assigned by the user. This alert will appear on the “buddies” phone similar to an Amber-Alert that needs to be turned off manually.

The “Save a Life, Link a Buddy” app will need to be downloaded by both the watch user and their assigned buddies. The application will run in the background and will be the home application that stores the users blood glucose history. Within the application, the user can assign “buddies,” which will then link the user and their buddies by phone number to allow the application to send alerts to all linked “buddies” if glucose levels drop or heighten to dangerous levels. During set up, the user will be required to set up the following specifications:
Link the application to their smart watch
Allow the application permission to transmit data (blood glucose levels) from the smart watch, allow sharing of location and to allow push notifications
Set their designated levels of “dangerous” blood glucose levels (both too high and too low)
Insert their medical information (age, sex, weight, insurance and medication information)
Assign “buddies”—individuals who also have the application downloaded on their phone. These buddies will then receive alerts when the wearer of the watch has blood glucose levels that reach dangerous levels and has not responded to their own alert from the app for over 60 seconds

“Buddies” will be required to set up the following specifications:
Allow the applications to send push notifications to their smart phone
Accept “buddy” requests if they want to receive notifications from a user
   How the “Save a Life, Link a Buddy” works:

If the user's glucose levels reach their designated “danger zone” the user will be presented with pulse checks to ensure they are conscious. The pulse checks will be in the form of simple puzzles that will appear on the user's smart phone (via the app). If the user cannot solve the puzzles or does not respond to the alert within 60 seconds their “buddies” will be sent a notification to their phone along with the user's GPS location. The designated buddy will be presented the option to call paramedics immediately or attend to the user if they are within a certain distance. If their glucose levels reach danger zones levels for over 5 minutes and no “buddies” note that they have responded in the app, paramedics will automatically be contacted with all of the user's medical information

The Diabetic Glucose Monitoring Smart Watch is a waterproof watch that will display glucose levels in addition to other common smart watch functionalities (time, other apps, etc.). The user will have the option to choose what information (glucose levels, time, heart rate, etc.) that will appear on their home screen. The watch will be connected via Bluetooth to both the user's smart phone and a waterproof sensor that will be located underneath a layer of tissue on the user's body, continuously monitoring glucose levels. If the individual does not wish to use a sensor, there will be a secondary option that allows the user to test their blood sugar manually and use the watch as a meter.

The watch will have additional features such as an A1C Estimator, Heart, Oxygen, Sleep, and Activity level Analysis available.

1. A Diabetic Glucose Monitoring Smart Watch Comprising: A Smart Watch, Glucose Meter, wherein the standard smart watch is converted to a glucose monitoring meter that will allow an individual to test their glucose reading and have it displayed in real time throughout the day.
A Diabetic Glucose Monitoring Smart Watch of claim 1 is a standard smart watch that is converted to a glucose monitoring meter that will allow an individual to test their glucose reading and have it displayed in real time throughout the day.
A Diabetic Glucose Monitoring Smart Watch of claim 2 is linked to a sensor that is attached via the arm and connected to the watch via Bluetooth connection.
A Diabetic Glucose Monitoring Smart Watch of claim 3 is a phone application that is linked to claims 1 and 2 and is connected via Bluetooth to the sensor and smart watch.
A Diabetic Glucose Monitoring Smart Watch of claim 4 is the strip and lancet storage compartments located on the wristband next to the face of the watch.
A Diabetic Glucose Monitoring Smart Watch of claim 5 is an alert system that will notify the user as well as the assigned “buddy/buddies” if the users glucose readings reach dangerous levels.
A Diabetic Glucose Monitoring Smart Watch of claim 6 is a medical dispatch feature that will not only contact medical personnel but allow them access to the users medical information and GPS location.
A Diabetic Glucose Monitoring Smart Watch of claim 7 is the feature of Customizable Target Zones that will allow a user to set specific ranges to help monitor their glucose levels for better A1C results.
A Diabetic Glucose Monitoring Smart Watch of claim 8 is a lancing device with interchangeable lancets that is stored and located on the base of the watch.
